(19) United States
(12) Plant Patent Application Publication
(10) Pub. No.: US 2003/0074706 P1
(43) Pub. Date: Apr. 17, 2003

(54) Chrysanthemum plant named "Scott"

(76) Inventor: Robert Noodelijk, Woubrugge (NL)

Correspondence Address: PARKHURST & WENDEL, SUITE 210, 1421 PRINCE STREET, ALEXANDRIA, VA, 22314, US

(21) Appl. No.: 09/923,392

(22) Filed: Aug. 8, 2001

(51) Int. Cl. A01H 5/00

(52) U.S. Cl. PLT/286

(57) ABSTRACT

A Chrysanthemum plant named ‘Scott’ characterized by its MEDIUM sized blooms with PURPLE ray-florets and YELLOW disc florets.

### BACKGROUND OF THE INVENTION

‘Scott’ is a product of a breeding-program which had the objective of creating new chrysanthemum cultivars with a daisy type flower, a 7 week response and a medium plant height. The new plant of the present invention comprises a new and distinct cultivar of Chrysanthemum plant. ‘Scott’ is a seedling from a cross in a breeding program maintained under the control of inventor. The female parent is #______ -unpatented-, an unamed seedling not available to inventor for description. The male parent is unknown, being a mixed pollation of a group of male parents. The new and distinct cultivar was discovered and selected as a flowering plant within the progeny of the stated cross by Rob Noodelijk in a controlled environment (greenhouse) in Rijsenhout Holland in ______ 19______.The first act of asexual reproduction of ‘Scott’ was accomplished when vegetative cuttings were taken from the initial selection in ______19______ in a controlled environment in Rijsenhout Holland.

### SUMMARY OF THE INVENTION

The present invention is a new and distinct variety of chrysanthemum bearing MEDIUM sized blooms with PURPLE ray-florets and YELLOW disc florets.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

FIG. 2 shows the various stages of bloom of the new cultivar.

FIG. 3 shows the foliage of the new cultivar

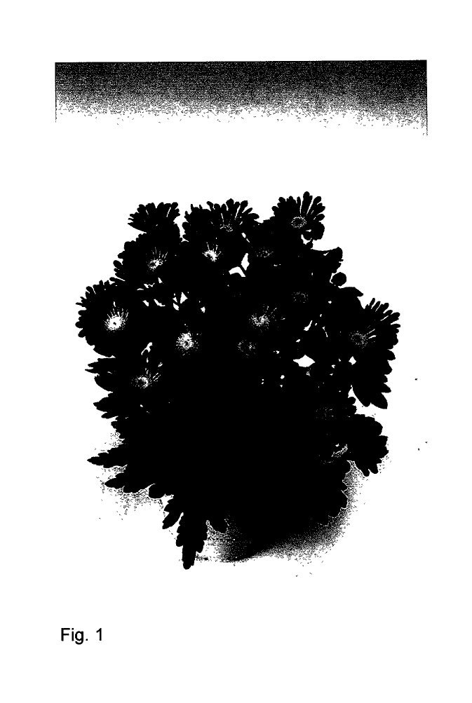

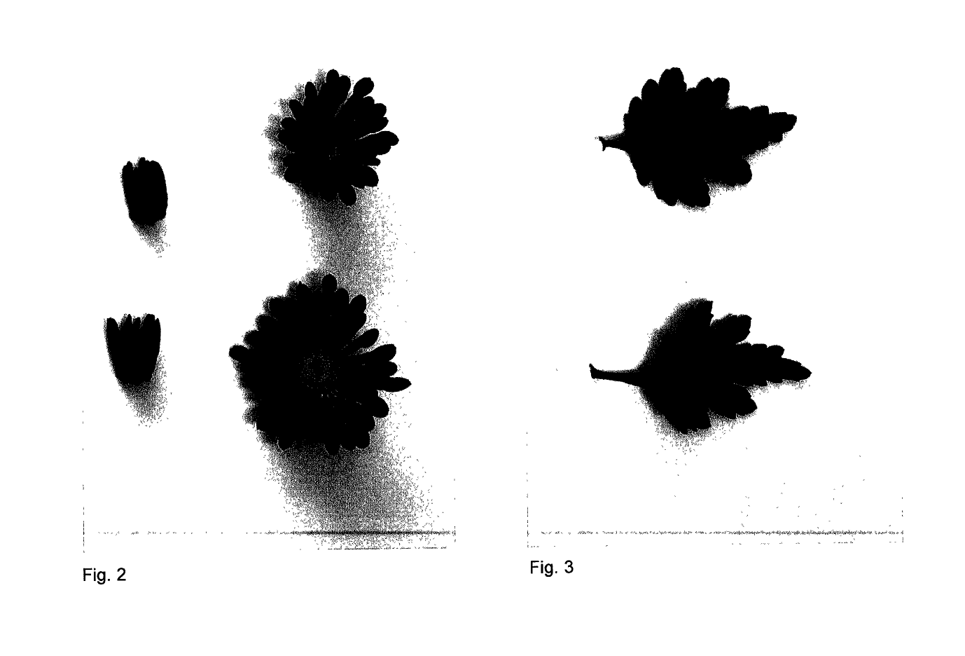

### DESCRIPTION OF THE INVENTION

This new variety of chrysanthemum is of the botanical classification dendranthema grandiflora. The observations and measurements were gathered from plants grown in a greenhouse in Rijsenhout Holland in a photo-periodic controlled crop under conditions generally used in commercial practice. The greenhouse temperatures during this crop were at day-time between 18° C. and 25° C. and at night 20° C. The photo-periodic response time in this crop was 50 days after an average of eight long days. After this long day period to flowering growth retardants were applied 6 times in an average dose of 1.5 gram/liter water. No tests were done on disease or insect resistance or susceptibility. This new variety produces MEDIUM sized blooms with PURPLE ray-florets and YELLOW disc-florets blooming on the plant for 5 weeks. This new variety of chrysanthemum has been found to retain its distinctive characteristics throughout successive propagations however the phenotype may vary significantly with variations in environment such as light intensity and temperature. To show the phenotype as described ‘Scott’ can be planted without assimilation lightning (high pressure sodium lamps) between week 50 and week 40 of the next year under greenhouse conditions in Holland. With assimilation lightning (minimum level 2500 lux) it can be planted year round under greenhouse conditions in Holland.

From the cultivars known to inventor the most similar existing cultivar in comparison to ‘Scott’ is ‘Rapture’. ‘Scott’ and ‘Rapture’ has a same type of daisy flower with spoontype ray-florets. When ‘Rapture’ and ‘Scott’ are being compared the following differences are noticed: The differences of ‘Rapture’ and ‘Scott’ are: (1) Flower color. The purple color of ‘Scott’ is darker. (2) Response time. The response time of ‘Scott’ is shorter. (3) Color of the disc florets. Center of the flower of ‘Scott’ is more yellow.

The following is a description of the plant and characteristics that distinguish ‘Scott’ as a new and distinct variety. The color designations are taken from the plant itself. Accordingly, any discrepancies between the color designations and the colors depicted in the photographs are due to photographic tolerances. The color chart used in this description is: The Royal Horticultural Society color chart, edition 1995.

### Table 1: Botanical Description of CULTIVAR ‘Scott’

Bud

Size.—medium;cross-section 1.0 cm, height 1.3 cm.

Outside color.—Red-purple 59 C.

Involucral bracts.—2 rows, length 7 mm, width 3 mm.

Involucral bracts among disc-florets.—Not present.

Involucral bracts color.—Green 138 A.

Bloom

Type.—Daisy.

Height.—Flat.

Size.—MEDIUM.

Fully expanded.—5.0-6.0 cm.

Number of blooms per branch.—Approx. 4 blooms per branch.

Performance on the plant.—5 weeks.

Seeds.—Not produced

Fragrance.—Typical chrysanthemum

Color

Center of the flower (disc-florets).—Immature a dot of yellow-green 144 A, most is yellow-green 151 A Mature yellow-green 151 A.

Color of upper surface of the majority of the ray-florets.—Red-purple 60 A.

Color of the lower surface of the majority of the ray-florets.—Red-purple 64 C.

Tonality from Distance.—A pot mum with PURPLE spoon type flower

Discoloration to color.—None

Ray florets

Texture.—Upper and under side smooth

Number.—39-41

Cross-section.—Tubular

Longitudinal axis of majority.—Straight

Length of corolla tube.—Long (1.5 cm.)

Ray-floret length.—2.5 cm.

Ray-floret width.—0.3 cm.

Ratio length/width.—High

Shape of tip.—Spatulate

Disc florets

Disc diameter.—1.5 cm.

Distribution of disc florets.—Numerous and clearly visible at all stages of flowering.

Shape.—Tubular.

Color.—Yellow-green 151 C.

Receptacle shape.—Conical raised.

Reproductive Organs

Stamen (present in disc florets only).—Thin, 4 mm. length.

Stamen color.—Yellow-green 151 C.

Pollen.—Present.

Pollen color.—Yellow 12 A.

Styles (present in both ray and disc florets).—Thin.

Style color.—Yellow-green 144 C.

Style length.—5 mm.

Stigmas.—Yellow-green 144 C.

Stigma width.—1 mm.

Ovaries.—Enclosed in calyx

Plant

Form.—A pot mum meant for indoor use

Growth habit.—Spreading

Growth rate.—Rapid

Height.—28.0-31.0 cm.

Width.—27.0 cm.

Stem color.—Green 138 A.

Stem strength.—Medium.

Stem brittleness.—Absent.

Stem anthocyanin coloration.—Present with a thin layer of greyed-red.

Length of lateral branch.—From top to bottom 14.0 cm.

Lateral branch color.—Green 138 A.

Lateral branch, attachment.—Weak.

Branching (average number of lateral branches).—Good with 5 breaks after pinching.

Peduncle length.—2.0-3.0 cm.

Peduncle color.—Green 138 A

Flowering response(photo-periodic controlled crop, not natural season.—50 Days.

Foliage

Color.—Upper side green 137 A Under side green 138 A.

Size.—Small; length 7.0 cm, width 4.0 cm.

Quantity (number per lateral branch).—5-6.

Shape.—Oval.

Texture upper side.—Glabrous.

Texture under side.—Pubescent.

Ribs and veins upper side.—Ribs and veins well developed.

Ribs and Veins upper side.—Ribs and veins well developed.

Venation arrangement.—Palmate.

Shape of the margin.—Serrated.

Shape of base of sinus between lateral lobes.—Acute.

Margin of sinus between lateral lobes.—Converging.

Shape of base.—Asymmetric.

Apex.—Cuspidate.

TABLE 2
Differences with the comparison varieties

| | ‘SCOTT’ | ‘RAPTURE’ |
|---|---|---|
| Color of ray-florets | Red-purple 60 A | Red-purple 61 B |
| Color of disc-florets | Yellow-green 151 A | Yellow-green 144 A |
| Response time | 50 days | 56 days |

I claim:

1. A new and distinct variety of chrysanthemum plant as described and illustrated.
